(19) United States
(12) Patent Application Publication
(10) Pub. No.: US 2008/0045929 A1
(43) Pub. Date: Feb. 21, 2008

(54) Method and infusion tubing connection system for selective administration of medicaments in the body

(76) Inventor: David J. Birnbach, Miami, FL (US)

Correspondence Address: JONES, TULLAR & COOPER, P.C., P.O. BOX 2266 EADS STATION, ARLINGTON, VA, 22202, US

(21) Appl. No.: 11/892,072

(22) Filed: Aug. 20, 2007

Related U.S. Application Data
(60) Provisional application No. 60/838,439, filed on Aug. 18, 2006.

(51) Int. Cl. A61M 39/10

(52) U.S. Cl. 604/533

(57) ABSTRACT

A system or kit adapted for controlling access to an epidural catheter includes: (a) a male-ended epidural connector; (b) a female ended sterile syringe that attaches to that male connector, but not to standard IV ports, the syringe is preferably color-coded (e.g., bright blue); (c) A connector device configured to permit the female syringe to be attached to a standard hypodermic needle, if necessary; and (d) A female-ended (and color-coded or tinted) epidural catheter configured to fit on the male-ended epidural connector, but to nothing else.

### RELATED PATENT AND PENDING PATENT APPLICATION INFORMATION

This application claims priority to provisional patent application No. 60/838,439, filed Aug. 18, 2006, the entire disclosure of which is incorporated herein by reference.

### BACKGROUND OF THE INVENTION

1. Field of the Invention

The present invention relates to methods and apparatus for selectively administering a specific intravenous medicament or other fluid using catheters or the like.

2. Discussion of the Prior Art

Treating disease in the body often requires infusion of drugs, blood products, nutritional fluids or other fluids into the patient's venous or arterial system, the patient's peritoneal or epidural space or other locations within the patient's body. The fluid (e.g., medication) typically is introduced through detachable syringe, drains from a container positioned above the patient to feed under gravity or is delivered via an infusion pump. The fluid flows through tubing and into a catheter (a tiny hollow tube). The catheter and the fluid tubing are usually removably attached to one another by a conventional luer lock connection.

Epidural techniques are very popular for pain relief in obstetrics and for postoperative pain management. The most often used technique involves placing the distal end of an epidural catheter into the epidural space in the body and then connecting the catheter's proximal end to an infusion source providing a continuous infusion of local anesthetic optionally including a selected added medicament such as an opioid.

When a patient needs supplemental drugs, sometimes the infusion source is temporarily disconnected from the catheter's proximal end connection and a standard syringe is connected to the epidural catheter. During this procedure, a mistake may result in the wrong syringe being connected to the catheter.

Generally, epidural anesthesia is used for lower abdominal, pelvic and lower extremity surgery and for pain relief in certain chronic and acute conditions such as during childbirth. The epidural anesthetic is usually administered by inserting an epidural catheter into the epidural space located just outside the spinal cord. Customarily, a standard epidural catheter connector is then attached to the free end of the epidural catheter. Subsequent to this, a standard syringe containing a local anesthetic is connected directly to the epidural catheter connector, and a moderate amount of the local anesthetic is slowly injected so that it passes through the epidural catheter and into the epidural space, flooding nerves emerging from the spinal cord. The degree and level of numbness produced is directly proportional to the amount of local anesthetic injected. In some instances, in addition to the initial dose, a continuous epidural infusion of local anesthetics or narcotics is initiated. This continuous infusion can provide pain relief for several days and is accomplished by using an epidural pump, a pump bag containing the medication to be given, and also continuous epidural tubing which connects to the epidural catheter connector. Before receiving the epidural anesthetic, the patient customarily has an intravenous line inserted for administering fluids and other medications. This intravenous line often has in-line injection ports or stopcocks through which medications can be given. A standard syringe and needle is used to inject medications into the intravenous line port. Injection into a stopcock is accomplished by using a standard syringe without a needle, and so standard syringes are being used to inject medications both into an epidural catheter and into an intravenous line present in the same patient concurrently.

If the large quantity of local anesthetic intended for injection into the epidural catheter is accidentally injected into the intravenous line, severe neurologic and cardiovascular problems, can occur. Alternatively, if some medications that are intended for intravenous use are accidentally injected into the epidural space, the patient may suffer severely.

In the past, many errors occurred because anesthesiologists were using the standard intravenous infusion tubing for epidural infusions and the tubes' connection ports could easily be mistaken for an intravenous port. Currently, this is not a major factor since many companies market special epidural tubing that does not have an infusion port. Errors, however, continue to occur.

For example, in the summer of 2006, a 16 year old girl died during childbirth under epidural when the wrong drug was administered. Unfortunately, the same syringes used in everyday practice for intravenous drug administration (male-ended) also fit into the connector of the epidural (female ended) so that occasionally an incorrect drug (one that might be neurotoxic—cause permanent nerve damage, or cardiotoxic-cause a cardiac arrest and death) is administered into the epidural space.

The “standard” syringes and “standard” connectors do not prevent these problems. A luer lock connection generally includes a male luer connector with a tapered conical portion adapted to fit into a correspondingly shaped receptacle of a female luer connector (or hub). A spin nut is commonly disposed on the male luer connector and is rotatable relative to the tapered conical portion. The spin nut includes internal threads adapted to engage external threads on the female luer connector to engage the luer connectors, locking them together. When properly engaged, the male luer's conical portion fits tightly within the female luer's receptacle to produce a sealed interconnection with aligned lumens in fluid communication with one another.

At present, the “standard” syringe has a male luer connector and “standard” connectors have a female luer fitting, and so IV bags, filter inlet connections and epidural catheter inlet connections all use female luer fittings.

Some practitioners have attempted to address the mechanism that makes the mistaken connection possible. For example, U.S. Pat. No. 5,616,133, to Cardenas, discloses a syringe for an epidural catheter having mechanical lockouts dimensioned to prevent IV medications from being administered into the epidural catheter. The Cardenas syringe includes a check valve and mates cooperatively with a fitting having a central valve actuating member and other features that may fail and that also require considerably more expense in manufacture and testing that the luer lock connector now in widespread use. The Cardenas syringes and connector are also an incomplete solution, in that prevention of human error relies solely on the mechanical features of the structure, and no added user-perceived indicia or procedural safeguards are included to ensure that users can confirm whether the correct infusate is being administered, before and during administration.

Others have proposed more straightforward solutions, for example, in the journal Anesthesia, (Number 57 of 2002, at pages 567-571, C. J. Lanigan, “Apparatus, Safer epidural and spinal connectors”) the author proposed reversing the luer lock connectors for epidural and spinal systems, using detachable connectors that may be improperly assembled. This journal letter is also silent about user-perceived indicia or procedural safeguards to ensure that users can confirm whether the correct infusate is being administered, before and during administration. This writer's approach seems to have been overlooked, since there are not, to the applicant's knowledge, any female ended syringes available for use in medical applications.

There is a need, therefore, for a convenient, safe, reliable, inexpensive and unobtrusive system and method for avoiding epidural infusion port related medical errors and their disastrous outcomes.

### OBJECTS AND SUMMARY OF THE INVENTION

Accordingly, it is an object of the present invention to overcome the above mentioned difficulties by providing a convenient, safe, reliable, inexpensive and unobtrusive system and method for preventing “wrong syringe” mistakes when making connections and infusing or injecting medicaments.

In accordance with the present invention, in order to prevent future such complications, the present invention comprises a new administration system for epidurals. Epidural catheters are configured so that visible indicia clearly and continuously show which syringe or connector should be used for an epidural catheter, and so that a standard syringe no longer fits in the epidural catheter's connector. Similarly, in accordance with the method and apparatus of the present invention, a syringe or other infusing tool filled with medicament for an epidural can no longer fit on an intravenous line.

As noted above, prior art epidural catheters resemble other catheters and are connected via a female luer connector that allows either a syringe or tubing to be connected. Prior art medical syringes marketed in the US all have male luer connectors and so fit into the female opening of the prior art epidural catheter. Female ended syringes are manufactured (e.g., for use as marinade flavor injectors and for use in the food service industry) but have not been available as sterile equipment for medical use and not marketed in the US.

Syringe bodies preferably also include color-coded visible indicia (e.g., blue writing with “Epidural Only”), whereas all prior art epidural syringes are clear.

In one embodiment of the present invention, a new epidural connector is manufactured to have a male ending (instead of the standard/prior art female ending) and a female syringe is made to fit the epidural connector's male ending, thereby dramatically reducing the risk of drug error involving epidural administration of drugs. Since a standard male syringe would not fit on the female end epidural connector, a standard intravenous drug could not be injected into the epidural space.

Similarly, epidural infusion tubing is made to have a female end to fit on the end of the epidural, but not into an intravenous line. A kit made in accordance with the present invention therefore includes the following:

1. A male-ended epidural connector;

2. A female ended syringe that attaches to that male connector but to nothing else. This syringe is color-coded bright blue; preferably, the stopper is colored, and the syringe's tubular body is translucent or clear;

3. A connector configured to permit the female syringe to be attached to a standard hypodermic needle, if necessary; and

4. A female-ended (and color-coded or tinted) epidural catheter configured to fit on the male-ended epidural connector, but to nothing else.

The kit of the present invention is preferably packaged as a unit and the kit optionally includes pre-packaged epidural infusion container clearly labeled (“Epidural Only”) and color coded (e.g., blue), where the epidural infusate container has a male ended epidural connector, optionally carrying a color-coded spin nut.

The syringe having the female luer connector has a unitary, one-piece cylindrical body with a female luer connector integrally affixed on its distal end, and is not provided in the form of a connector adapted for attachment to a standard tapered end syringe.

The above and still further objects, features and advantages of the present invention will become apparent upon consideration of the following detailed description of a specific embodiment thereof, particularly when taken in conjunction with the accompanying drawings, wherein like reference numerals in the various figures are utilized to designate like components.

### DESCRIPTION OF THE DRAWINGS

FIG. 1 illustrates the standard male luer-ended syringe of the prior art.

FIG. 2 illustrates the standard infusion tubing set with a male luer connector in accordance with the prior art.

FIG. 3 illustrates the standard male luer-ended syringe's connection to a standard female ended needle, in accordance with the prior art.

FIG. 4 illustrates the standard female luer-ended epidural connector, adapted to receive the standard male infusion tube or syringe, in accordance with the prior art.

FIG. 5 illustrates a new epidural syringe including a cylindrical hollow body terminating distally in a female luer end and receiving an elongated plunger having a proximal thumb pad and terminating distally in a sliding stopper or seal, in accordance with the present invention.

FIG. 6 illustrates a new epidural catheter including a selected length of catheter tubing terminating distally in a male luer end carrying, optionally, a color coded spin nut, in accordance with the present invention.

FIG. 7 illustrates the differences between the new male-luer ended connector and the standard connector, in accordance with the present invention.

FIG. 8 illustrates the new infusion tubing set with a female luer connector, in accordance with the present invention.

FIG. 9 illustrates an adapter optionally included as part of the kit of the present invention; the adapter has first and second opposing and coaxially aligned male luer connectors in fluid communication with a common channel, for use when connecting the new female ended syringe to a standard needle (e.g., when filling the syringe), in accordance with the present invention.

FIG. 10 illustrates another embodiment of the new epidural syringe including a cylindrical hollow body terminating distally in a female luer end and receiving an elongated color-coded plunger, in accordance with the present invention.

### DESCRIPTION OF THE PREFERRED EMBODIMENT

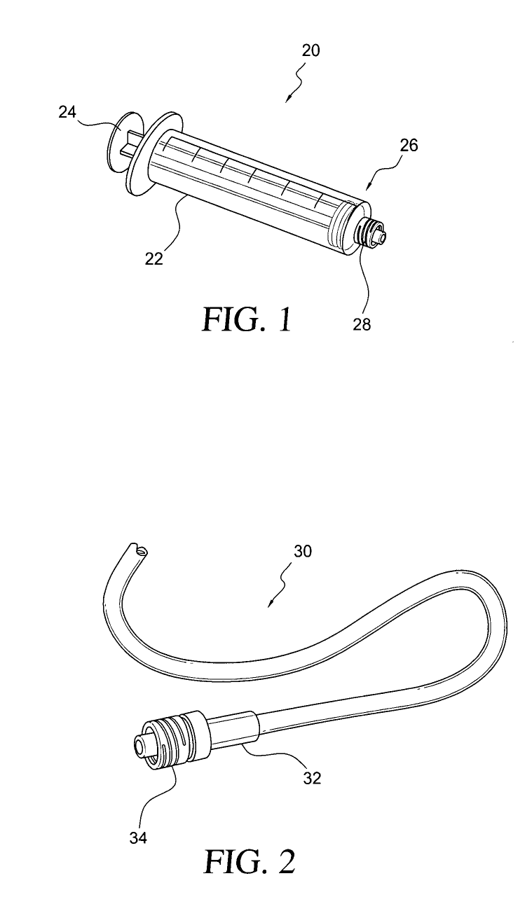

Referring now to FIGS. 1-10, in accordance with the present invention, in order to prevent mistakes (in choosing the correct syringe or other infusion source) when administering therapeutic fluids or medicaments, the present invention comprises a new administration system (e.g., for epidurals).

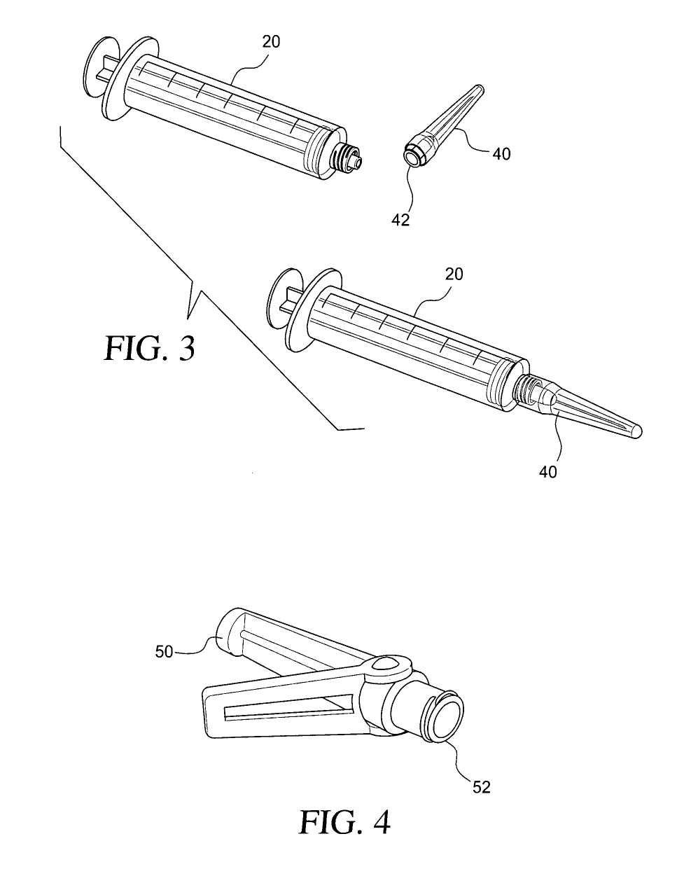

For purposes of nomenclature, the prior art or standard implements will be described. Referring to FIGS. 1-4, FIG. 1 illustrates standard syringe 20 having cylindrical hollow body 22 which receives a sealed, sliding plunger 24 which terminates distally in seal or stopper 26. Syringe body 22 terminates distally in male luer connector 28. FIG. 2 illustrates the standard infusion tubing set 30 with a male luer connector 32 that may carry a spin nut 34. FIG. 3 shows the connection of standard male luer-ended syringe 20 to a standard needle 40 supported from a proximal female luer connector 42. FIG. 4 illustrates a standard epidural connector 50 with a female luer end 52, which is adapted to receive the standard male infusion tube or syringe 20.

In accordance with the method and apparatus of the present invention, a new or non-standard design for an epidural catheter connector 70 (FIG. 6) is provided so that standard syringe 20 no longer fits in the epidural catheter's connector 74 and similarly so that syringe 20 or some other standard infusing tool filled with medicament for an epidural can no longer fit on an intravenous line. Currently, all epidural catheters are connected via a female connection device that allows either a syringe or tubing to be connected. All medical syringes marketed in the US are male-ended and so fit into the female opening of the epidural catheter.

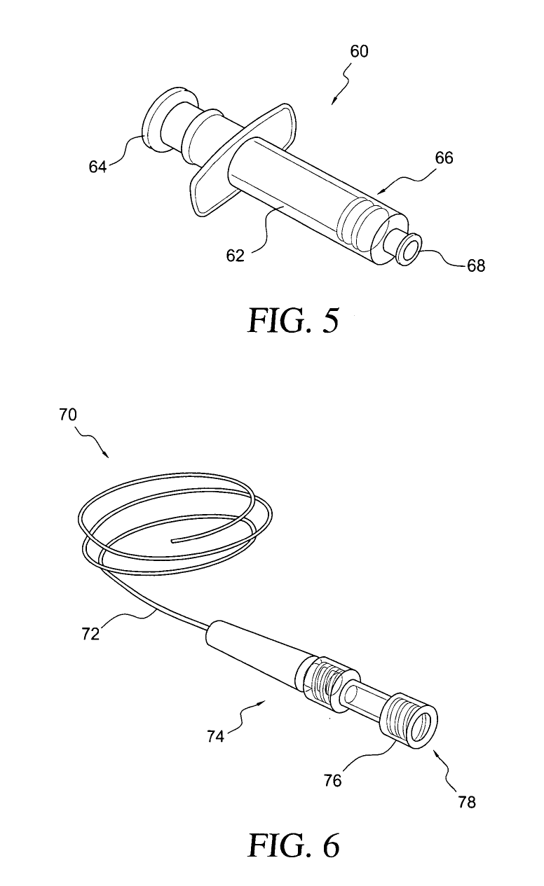

Referring again to FIG. 5, new epidural syringe 60 includes a polymer, disposable, sterile, one-piece cylindrical hollow body 62 terminating distally in an integral female luer end 68 and receives an elongated sterile plunger 64 having a proximal thumb pad and terminating distally in a color coded (e.g., blue) sliding stopper or seal 66. Syringe 60 is preferably sized to provide measured doses in selected quantities (e.g., 10 ml and 25 ml).

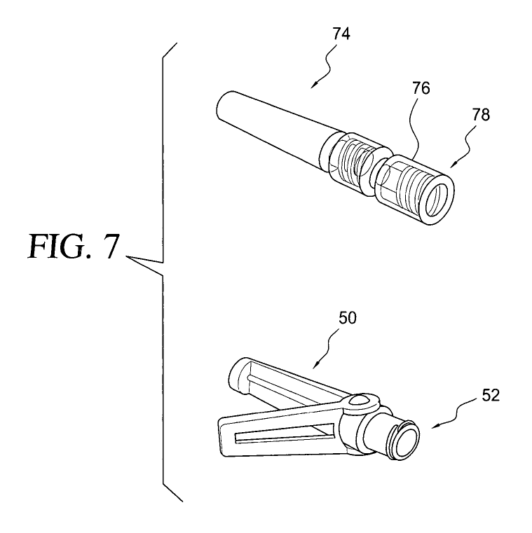

FIG. 6 illustrates new epidural catheter set 70 including a selected length of sterile catheter tubing terminating distally in a permanently affixed male luer end 74 carrying, optionally, a color coded spin nut 76. Spin nut, is preferably tinted or colored blue, to provide users additional confirmation of the proper connection points, when making the connection. The color coding also provides confirmation of correct administration after connections are made. FIG. 7 illustrates the differences between the new male-luer ended connector 74 and the standard connector 50.

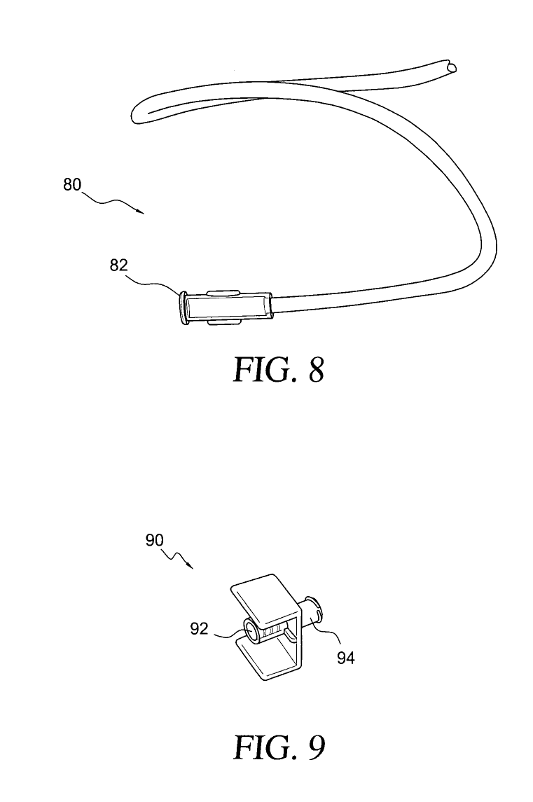

FIG. 8 illustrates a sterile, disposable infusion tubing set 80 with a permanently affixed female luer connector 82.

FIG. 9 illustrates an adapter 90 optionally included as part of the kit of the present invention; adapter 90 has first and second opposing and coaxially aligned male luer connectors 92, 94 in fluid communication with a common axial channel, for use when connecting the new female ended syringe (e.g., 60 or 116) to standard needle 42, for example, when filling syringe 60.

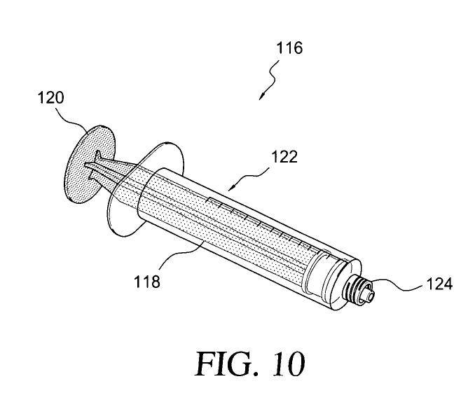

FIG. 10 illustrates another embodiment of a new epidural syringe 116 including a cylindrical hollow body 118 terminating distally in a female luer end 124 and receiving an elongated color-coded plunger 120 having color coded sides visible through the translucent side wall of body 118 and along the length of the plunger extending proximally from body 118 when plunger 120 is drawn back. Syringe 116 is also preferably sized to provide measured doses in selected quantities (e.g., 10 ml and 25 ml). Here again, color coding provides confirmation of correct administration before, during and after connections are made. Color coding and labeling of the components described above (FIGS. 6-10) provide user-perceived indicia that give medical professionals the ability to enact simple procedural safeguards, ensuring that users can confirm whether the correct infusate is being administered, before, during and after administration.

The embodiments illustrated in FIGS. 6-10 are prototypes, and so the invention contemplates finished items manufactured as one-piece components with permanently affixed luer connectors, and packaged as sterile, disposable components ready for use in medical practice. For enablement purposes, these prototypes will be described more plainly. Most of the components will be manufactured to order, since they were unavailable. For sterile, disposable colored syringes, especially with colored plungers or plunger seals, none are currently available. At present, sterile female-luer ended syringes are unavailable for medical use. The prototype female luer syringe depicted is not sterile and is manufactured only in 5 ml variety (and we would need 10 ml and 25 ml) by Qosina, Inc. Edgewood, N.Y. 11717. Part no. C3603.

The prototype male tip epidural connector 74 was made by taking the BBraun Tuohy Borst connector (B Braun, Bethlehem, Pa.) and permanently bonding on a female to male adapter. The prototype's adapter (female to male) is produced by Becton Dickinson, part 408299. While this illustrates how the prototype is made, in the preferred embodiment, new epidural connector 74 is preferably a one-piece, unitary male ended luer connector optionally including a color coded surface, and is sterile.

The prototype female epidural infusion catheter of FIG. 8 was made by modifying a standard B Braun catheter (male ended) and permanently bonding on a male to female converter (custom made- not currently manufactured). While this illustrates how the prototype is made, in the preferred embodiment, new infusion tubing set 80 is preferably comprises a catheter tubing segment permanently affixed to a one piece, unitary female ended luer connector optionally including a color coded surface, and is sterile.

It will be appreciated by those with skill in the art that while using this system provides greater safety, there is also the option of retaining backward compatibility with the standard connectors and catheters of the prior art. The apparatus of the present invention includes a non-standard male epidural catheter opening so that either new epidural infusion tubes are needed or an adapter can be added to convert the male infusion tuning to be a female infusion tubing (FIG. 8 shows the new epidural infusion tubing set 80 and FIG. 9 shows the adaptor 90 so that one can either (a) add a needle to the new female syringe (to help fill syringe with medications) or (b) use an old male-ended infusion tubing to the new male epidural (in case no new infusion tubes are available).

FIG. 5 shows the new female ended syringe. No such syringe is currently manufactured for medical use (one manufacturer makes it for industrial use, non-sterile). FIG. 6 shows the new male ended epidural connector that fits with the new female ended syringe. It is readily seen that a standard syringe (male) won't fit into this male epidural connector. FIG. 7 shows the new epidural connector (male ended-will only attach to a female syringe) versus a standard currently available epidural connector (shown on the right) which fits a standard syringe (hence the problem of injecting the wrong drug into the epidural. Since the epidural connector is now male, the infusion tip will need to be female. This prevented the occasional accidental attachment of an epidural infusion bag to an unknown Intravenous line, since it's a female end, it can't easily be attached to a needle and thus can't be attached to an IV. Adapter 90 of FIG. 9 allows one to occasionally convert the new male ended catheter to be a female (standard end), so that in an emergency one can switch back from the new to the old catheter. The color coding or blue color is easily recognizable and is to be limited only to epidural use so that no one can ever see the syringe and think the syringe contains IV medication, or pick up a standard clear syringe and inject it into the epidural space.

In one embodiment of the present invention, a new epidural connector is manufactured to have a male ending (instead of the standard/prior art female ending) and a female syringe is made to fit the epidural connector's male ending, thereby dramatically reducing the risk of drug error involving epidural administration of drugs. Since the standard male syringe of the prior art will not fit on the female end epidural connector, a standard intravenous drug cannot be injected into the epidural space through that connector.

Similarly, epidural infusion tubing is made to have a female end to fit on the end of the epidural, but not into an intravenous line.

A kit 130 made in accordance with the present invention therefore includes the following:

1. A catheter 70 carrying a permanently affixed male-ended epidural connector 74 (as in FIG. 6);

2. A female ended syringe (e.g., 60 or 116) that attaches to male connector 74 (but not to a standard IV line). Syringe 60 or 116 is preferably color-coded bright blue (e.g., for syringe 60 stopper or seal 66 is colored, the syringe's tubular body 62 is translucent or clear);

3. A connector device or adapter 90 (as in FIG. 9) configured to permit the female syringe to be attached to a standard hypodermic needle, if necessary; and

4. A female-ended (and color-coded or tinted) infusion tubing set 80 (as in FIG. 8) configured to fit on the male-ended epidural connector 74, but not a standard IV line).

It will be appreciated by those of skill in the art that the system and method of the present invention provides a solution to problems associated with mistakes in situations where a plurality of infusates or therapeutic fluids are administered through a plurality of connectors (e.g., luer locks), ends or ports.

Broadly speaking, the system of the present invention includes, a catheter 70 or other fluid passage into the body having a tubular member 72 with distal end adapted to be inserted in the body and a proximal end in fluid communication therewith; the catheter proximal end is color coded carries a first catheter connector 74 configured to releasably connect with a first mating connector (68 or 124). The system (or kit 130) also includes a first syringe (60 or 116) carrying a first fluid selected for infusion into the catheter 70, where the first syringe (60 or 116) has the first mating connector configured to connect with the catheter connector 74 in a manner permitting the first syringe's first fluid to be infused into the catheter 70 through the catheter connector. First syringe 60 or 116 is color coded to match the catheter's proximal end. The system may be used in a patient care environment in which staff often use a second (standard) syringe 20 carrying a second fluid (such as IV medications), where the second fluid was not selected for infusion into the catheter 70, and where the second syringe 20 has the second connector 28, where the second connector differs from the first and so is not configured to connect with the catheter's connector 74; and where the second syringe's second fluid is thereby prevented from being infused into the catheter 70 through the catheter connector 74. Second syringe 20 is not color coded or color matched to first syringe (60 or 116) and so is visually distinguishable from the first syringe and the catheter's connector 74.

In the illustrated embodiments, the second syringe's second connector 28 is a standard male-ended luer connector and the first syringe's mating connector (e.g., 68) is a female-ended luer connector. Also, the system's catheter's first connector is a male-ended luer connector.

As noted above, the first syringe carries a visually distinguishable indicia and so is readily distinguished from a standard syringe (e.g., it is color-coded) and, in accordance with the method of the present invention, the color coding and application-specific labeling of the components provide user-perceived indicia that give medical professionals the ability to enact simple procedural safeguards, ensuring that users can confirm whether the correct infusate is administered before, during and after administration.

Having described preferred embodiments of a new and improved method and apparatus, it is believed that other modifications, variations and changes will be suggested to those skilled in the art in view of the teachings set forth herein. It is therefore to be understood that all such variations, modifications and changes are believed to fall within the scope of the present invention.

What is claimed is:

1) A system for administering medicaments or other therapeutic fluids to a patient by infusion, including:
a sterile catheter having a tubular member with distal end adapted to be inserted in the body and a proximal end in fluid communication therewith;
said catheter proximal end carrying a first catheter connector configured to releasably connect with a first mating connector;
said catheter proximal end carries a visually distinguishable indicia and so is readily distinguished from a standard catheter connection;
a first sterile syringe or infusate source carrying a first fluid selected for infusion into said catheter, said first syringe having said first mating connector configured to connect with said catheter connector in a manner permitting said first syringe's first fluid to be infused into said catheter through said catheter connector; and
wherein said system is adapted for use in a patient care environment also including a second syringe carrying a second fluid, said second fluid not being selected for infusion into said catheter, said second syringe having said second connector, said second connector being incompatible for connection with said catheter connector; and
wherein said second syringe's second fluid is prevented from being infused into said catheter through said catheter connector.

2) The system of claim 1, wherein said second syringe's second connector is a standard male-ended luer connector.

3) The system of claim 1, wherein said first syringe's mating connector is a female-ended luer connector.

4) The system of claim 3, wherein said catheter's first connector is a male-ended luer connector.

5) The system of claim 1, wherein first syringe carries a visually distinguishable indicia and so is readily distinguished from a standard syringe.

6) The system of claim 5, wherein first syringe carries a visually distinguishable indicia matching said catheter indicia and so is readily distinguished by color from a standard syringe.

7) The system of claim 6, wherein said visually distinguishable indicia is a color coded marking signifying that said catheter connector and said first syringes are intended for use solely with one another.
